(19) United States
(12) Patent Application Publication
(10) Pub. No.: US 2022/0347456 A1
(43) Pub. Date: Nov. 3, 2022

(54) Fiber Optic Disinfection Device

(71) Applicant: Bard Access Systems, Inc., Salt Lake City, UT (US)

(72) Inventor: Shayne Messerly, Kaysville, UT (US)

(21) Appl. No.: 17/732,614

(22) Filed: Apr. 29, 2022

Related U.S. Application Data
(60) Provisional application No. 63/182,363, filed on Apr. 30, 2021.

(51) Int. Cl. A61M 39/16; A61L 2/00; A61M 25/00

(52) U.S. Cl. CPC A61M 39/16; A61L 2/0047; A61M 25/0017; A61M 25/0045; A61L 2202/24; A61M 25/0023

(57) ABSTRACT

A system for disinfecting a medical device is disclosed that includes an elongate instrument comprising a plurality of optical fibers extending along a length of the instrument to a disinfection zone, and a light source coupled with the instrument configured to propagate light distally along the optical fibers. The elongate instrument can be configured to redirect the light radially outward from the instrument. An elongate instrument for disinfecting a medical device can include a plurality of optical fibers extending along a length of the instrument from a proximal end to a disinfection zone at a distal end, the optical fibers configured to propagate a light along the instrument. One or more reflective surfaces can be located within the disinfection zone, and the reflective surfaces can be configured to direct the light radially outward from the instrument.

### PRIORITY

This application claims the benefit of priority to U.S. Provisional Application No. 63/182,363, filed Apr. 30, 2021, which is incorporated by reference in its entirety into this application.

### BACKGROUND

The use of UV light as a disinfection means for surfaces is common. However, the effectiveness of UV light for disinfection is often limited due to difficulty in effectively delivering the UV light at the required intensity to the desired locations. In some instances, tubular devices such as catheters may become contaminated during use, which in some instances may require replacement of the catheter. Some catheters, such central venous catheters, may be remain inserted within the patient for an extended period of time making them more susceptible to contamination. Central venous catheters may also require a complex process for replacement that puts the patient at risk. The relatively high contamination rate and the complexity of the replacement process combined with the risk to the patient, create a need for disinfecting such catheters while the catheter remains inserted. Disclosed herein are systems, devices, and methods for disinfecting tubular medical devices.

### SUMMARY

Briefly summarized, disclosed herein is a system for disinfecting a medical device, including an elongate instrument including a plurality of optical fibers extending along a length of the instrument from a proximal end to a disinfection zone at a distal end, and a light source operatively coupled with the instrument at the proximal end.

The instrument is configured for insertion into a medical device, where the medical device is an elongate tubular medical device. The medical device may be a catheter such as a vascular catheter. The instrument may be configured for insertion into the medical device while the medical device is inserted within a patient body.

The instrument defines a circular cross section and the instrument may include a conical reflective surface within the disinfection zone. In some embodiments, each optical fiber includes the reflective surface within the disinfection zone. The reflective surface may be configured to direct fiber optic light radially outward from the instrument. In some embodiments, a wavelength range of the light extends only between 100 nm and 400 nm.

The instrument may include a sheath extending along the length, where the sheath is formed of a material transparent to ultra-violet light.

The instrument may include indicia disposed on the instrument along a least a portion of the length, and the indicia may include graduation marks.

The system may include a timer configured to provide an alert at the conclusion of a defined time period. The instrument may include a handle disposed at the proximal end, and the handle is configured for manipulation of the instrument by a clinician.

The system may further include an interconnect extending between the instrument and the light source, the interconnect including interconnecting optical fibers to facilitate propagation of the light between the light source and the instrument.

Also disclosed herein is an elongate instrument for disinfecting a medical device. The instrument includes a plurality of optical fibers extending along a length of the instrument from a proximal end to a disinfection zone at a distal end, where the optical fibers are configured to propagate a light along the instrument. The instrument further includes one or more reflective surfaces located within the disinfection zone, where the reflective surfaces are configured to direct the light radially outward from the instrument. The instrument additionally includes a connector at the proximal end configured to couple with a light source.

The instrument is configured for insertion into a medical device, which may be a tubular medical device. The medical device may also be a catheter, such as a vascular catheter. The instrument may also be configured for insertion into the medical device while the medical device is inserted within a patient body. The instrument may also define a circular cross section.

The instrument may include a core extending between the proximal end and the disinfection zone and a sheath extending between the proximal end and the disinfection zone, where the sheath is disposed over the core so as to cover the core. The sheath may be transparent to ultra-violet light.

In some embodiments, the optical fibers are disposed within the core, and the optical fibers may be disposed adjacent a circumferential surface of the core.

In some embodiments, the instrument includes a plug coupled with the core at a distal end of the core, and the plug includes a conical reflective surface. In such an embodiment, the conical reflective surface defines the one or more reflective surfaces.

In some embodiments, each optical fiber includes a cavity extending laterally across an optical core of the optical fiber, where the optical core is configured to propagate the light along the optical fiber, and a wall of the cavity is a reflective surface configured to direct the light from the optical core radially outward from the instrument.

In some embodiments, the core includes one or more notches disposed on the circumferential surface along the disinfection zone. The notches extend inward to the optical fibers, and the notches are configured for passage of light therethrough.

The instrument may include graduation marks disposed on the instrument along a least a portion of the length and a handle disposed at the proximal end, where the handle is configured for manipulation of the instrument by a clinician.

Also disclosed herein is a method for disinfecting a tubular medical device. The method includes providing an elongate instrument, where the instrument includes a plurality of optical fibers extending along a length of the instrument from a proximal end to a distal end, the optical fibers configured to propagate a light along the instrument. The method further includes, coupling the instrument with an ultra-violet light source, inserting the instrument into a lumen of the medical device, activating the light source, and impinging the light onto an inside luminal surface of the medical device.

In some embodiments of the method, the instrument further includes a disinfection zone disposed at the distal end, and the disinfection zone includes one or more reflective surfaces configured to direct the light radially outward from the instrument.

The method may further include positioning the disinfection zone at a desired location within the lumen, and adjusting the position of the disinfection zone to different location within the lumen.

The method may further include deactivating the light source and removing the instrument from the medical device.

In some embodiments, activating the fiber optic light source is performed after inserting the instrument into a lumen of the medical device, and deactivating the fiber optic light source is performed before removing the instrument from the medical device.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an illustration of exemplary embodiment of a disinfection system for tubular medical device, in accordance with some embodiments;

FIG. 2 illustrates cross section of a disinfecting instrument of the system of FIG. 1;

FIG. 3 illustrates detail cross-section side view of a first exemplary embodiment of the disinfection zone of FIG. 1, in accordance with some embodiments; and

FIG. 4 illustrates detail cross-section side view of a second exemplary embodiment of the disinfection zone of FIG. 1, in accordance with some embodiments, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The directional terms “proximal” and “distal” are used herein to refer to opposite locations on a device or instrument. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

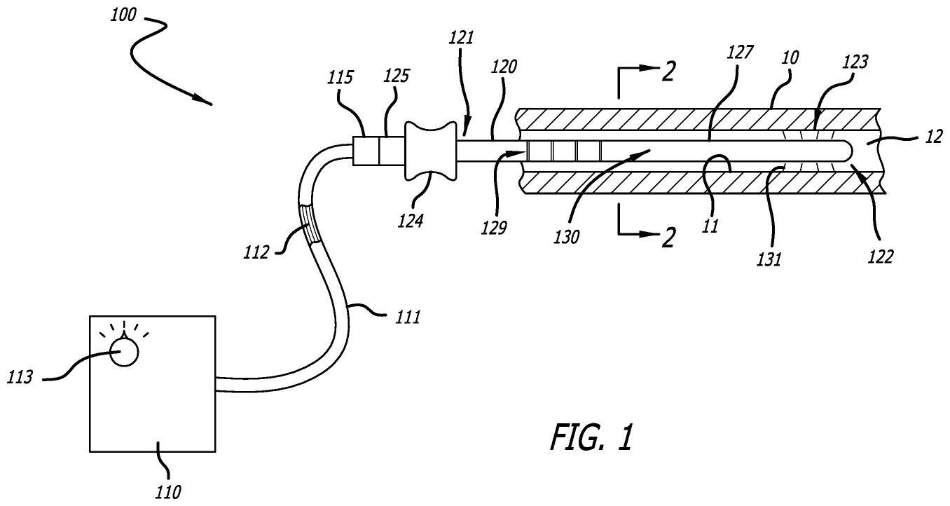

FIG. 1 is an illustration of exemplary embodiment of a disinfection system 100 for a tubular medical device 10, in accordance with some embodiments. The system 100 is configured to disinfect an inside surface 11 of a lumen 12 of the medical device 10 by impinging a light 131 within the ultra-violet (UV) spectrum onto the inside surface 11. The medical device 10 may be a vascular catheter, a drainage catheter, or any other tubular medical device. The system 100 generally includes a disinfecting instrument 120 operatively coupled with a light source 110. The instrument 120 includes a plurality of optical fibers 130 extending between a proximal end 121 of the instrument 120 and a disinfection zone 123 adjacent a distal end 122 of the instrument 120. The instrument 120 is operatively coupled with the light source via an instrument connector 125 in combination with a light source connector 115. The instrument 120 may include a handle 124 to facilitate manipulation of the instrument 120 by a clinician.

The system 100 may include an interconnect 111 extending between the light source 110 and the instrument 120. The interconnect 111 includes interconnecting optical fibers 112 to facilitate propagation of the light 131 between the light source 110 and the optical fibers 130 of the instrument 120. In general terms, the interconnect 111 may be a flexible fiber-optic cable having a sufficient length (e.g., from about two to ten feet) to extend between a benchtop light source 110 and the medical device 10 which may be inserted into a patient. In some embodiments, the instrument 120 may be directly connected to the light source, in which embodiments, the interconnect 111 may be omitted. In such embodiments, the light source 110 may also define the handle 124 of the instrument 120.

The instrument 120 is sized for insertion within the lumen 12 of the medical device 10. As such, the instrument 120 may include stiffness and flexibility characteristics similar to a guidewire. As the instrument 120 may be inserted into a vascular medical device while the medical device is inserted into a vasculature of the patient, the instrument 120 is configured for advancement along the medical device 10 when the medical 10 is shaped (i.e., curved) to follow the vasculature. In some embodiments, the instrument 120 may be sterilized prior to use.

The instrument 120, along the disinfection zone, is configured to provide for exposure of the light 131 propagating through and long the optical fibers 130, onto the inside surface 11 of the medical device 10. The instrument 120 may employ reflection, refraction, dispersion, curving of optical fibers, or any other mechanism for redirecting the light 131 from a longitudinal direction within the optical fibers 130 to a radially outward direction.

In some embodiments, a cross-sectional size or dimension of the instrument 120 may define a minimal clearance between an outside surface 127 of the instrument 120 and the inside surface 11 of the lumen 12. The minimal clearance may inhibit degradation of the intensity of the light 131 impinging onto the inside surface 11. A length of the instrument 120 may be defined to facilitate advancement of the disinfection zone 123 to a distal end of the medical device 10. As medical devices vary in size, multiple sizes of the instrument 120 may be provided to accommodate multiple sizes of the medical device 10.

The instrument 120 may include indicia 129 disposed along its length. The indicia 129 may be configured to indicate a position (i.e., rotational and/or longitudinal) of the instrument 120 within the medical device 10. In some embodiments, the indicia may include one or more graduation marks indicative of a distance to the disinfection zone 123. A spacing of the graduation marks may be consistent with an effective length of the disinfection zone 123. In use, the clinician may insert the instrument 120 into the medical device 10 until a desired graduation mark is adjacent a proximal end of the medical device 10. Thereafter, the clinician may adjust the position of the instrument 120 so that another graduation mark is adjacent the proximal end of the medical device 10. By repeatingly adjusting the position of the disinfection zone 123, the system 100 may effectively disinfect the entire inside surface 11 (or a desired portion thereof) of the medical device 10.

The light source 110 is configured to provide light 131 to the instrument 120 within the UV spectrum. As such, the light 131 may include wavelengths ranging between about 100 nm and 400 nm. In some embodiments, the wavelengths of the light 131 may be between about 100 nm and 400 nm, 100 nm and 280 nm, 240 nm and 280 nm, or 260 nm and 270 nm. The light source 110 may include a laser, a light emitting diode (LED), or any light source suitable for emitting UV light within the defined wavelength range. The light source 110 may include other supporting components including a power supply, switches, indicator lights, lenses, fuses, cords, electric wiring or cables, a housing, and any other functionally supporting components. In some embodiments, the light source 110 may be powered via an internal battery.

In some embodiments, the instrument 120 (or more specifically the light source 110) may include a timer 113 which in some embodiments may be adjustable. The timer 113 may be configured to provide an alert (e.g., an audio alert) at the conclusion of a desired time period. In some embodiments, the time period may align with an effective disinfection duration. In use, the clinician may position the instrument 120 at a desired location within the medical device 10 and activate the timer 113. Upon receiving the alert, the clinician may position the instrument 120 at a different desired location within the medical device 10 and reactivate the timer 113.

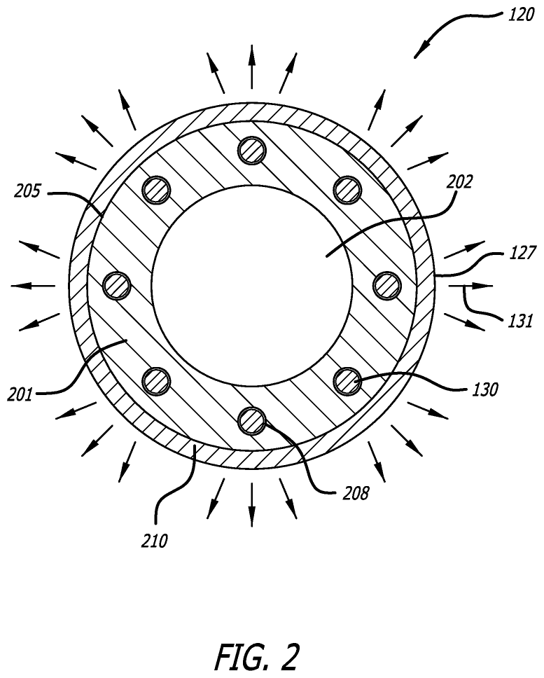

FIG. 2 is cross-sectional view of the instrument 120 cut a lot sectioning lines 2-2 as shown in FIG. 1. The instrument 120 includes a core 201 extending along the length of the instrument 120. In some embodiments, the core 201 may have a tubular structure including a lumen 202. In other embodiments, the core 201 may have a solid cross section (i.e., the lumen 202 may be omitted). The optical fibers 130 extend along the core 201 and may be positioned adjacent an outside circumferential surface 205 of the core 201 along at least a portion of the length of the instrument 120. The core 201 may be formed of a plastic material via the extrusion process. The material may be polypropylene, polyethylene, polyvinylchloride, polytetrafluorethylene, or any other suitable plastic material. In some embodiments, micro lumens 208 may be formed along the core 201 during the extrusion process after which the optical fibers 130 may be inserted within the micro lumens 208. In other embodiments, the optical fibers 130 may be inserted within the core 201 during the extrusion process.

The instrument 120 may include a sheath 210 providing a covering for the core 201 and the sheath 210 may extend between the proximal end 121 and the distal end 122 of the instrument 120. The sheath 210 may be formed of any material suitably transparent to the UV light 131, such as fluorinated ethylene propylene (FEP Teflon), for example.

As illustrated in FIG. 2, the instrument 120 may include eight optical fibers 130. In other embodiments, the instrument 120 may include more or less than eight optical fibers 130. In some embodiments, the circumference of the core 201 may be entirely lined with optical fibers 130 such circumferential spacing between adjacent optical fibers 130 is minimized. Such an embodiment may maximize the transmission of light 131 along the instrument 120 to the disinfection zone 123.

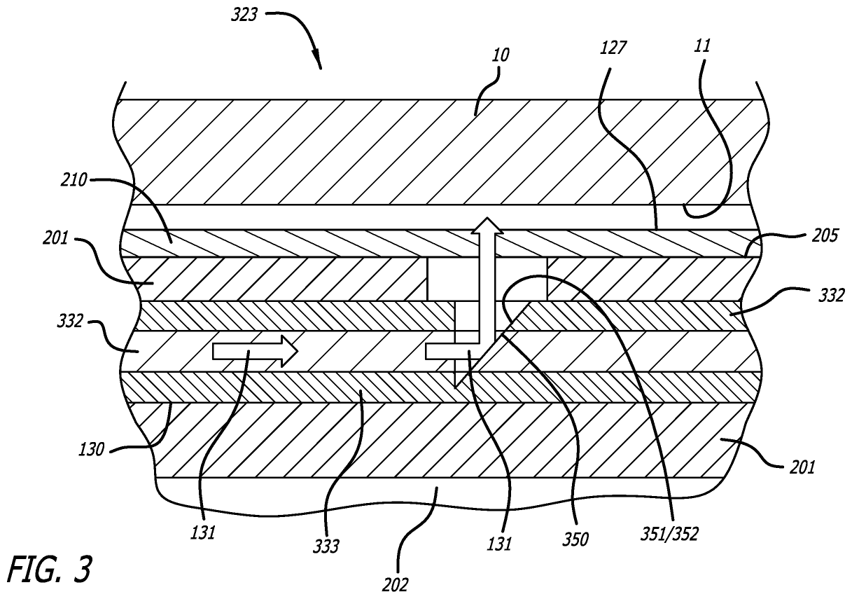

FIG. 3 is a detail cross-sectional view of one embodiment of the disinfection zone of FIG. 1. A disinfection zone 323 shown in conjunction with the medical device 10. As shown, the inside surface 11 of the medical device 10 is disposed opposite the outside surface 127 of the instrument 120 (i.e., the outside surface of the sheath 210). The sheath 210 is disposed on the outside surface of the core 201, and the optical fiber 130 (i.e., one of the plurality of optical fibers 130) is disposed adjacent the circumferential surface 205 of the core 201. The optical fiber 130 includes an optical core 332 disposed within a cladding 333 and the light 131 propagates longitudinally through the optical core 332.

A notch 340, formed in the core 211, extends radially inward to the optical fiber 130. The notch 340 is closed on the top by the sheath 210. The notch 340 provides an aperture for light 131 to pass through as described below. In some embodiments, a separate notch 340 may be formed for each optical fiber 130. In other embodiments, a single annular notch (e.g., an annular groove) 340 may extend inward to each optical fiber 130.

A cavity 350 formed in the optical fiber 130 extends across the optical core 332. The cavity 350 includes angled surface 351 having a reflective material 352 disposed thereon. In use, the light 131 propagates along the optical core 332, exits the optical core 332, and enters the cavity 350. The light 131 reflects off the angled surface 351, so that the light 131 is directed radially outward. The light 131 passes through the notch 340 and the sheath 210, after which the light 131 impinges onto the inside surface 11 of the medical device 10.

A similar cavity 350 as described above may be formed in each optical fiber 130 so that the light of each fiber 130 is directed to impinge onto the inside surface 11. As such, the disinfection zone 123 defines an annular ring of light 131 impinging onto the inside surface 11. In some embodiments, the cavities 340 of adjacent optical fibers 130 may be longitudinally offset longitudinally expand the annular ring of light 131 impinging onto the inside surface 11, thereby forming an increased length of the disinfection zone 123. As may be appreciated by one of ordinary skill, other arrangements of cavities 340 may further increase or otherwise maximize the effective area of the disinfection zone 123.

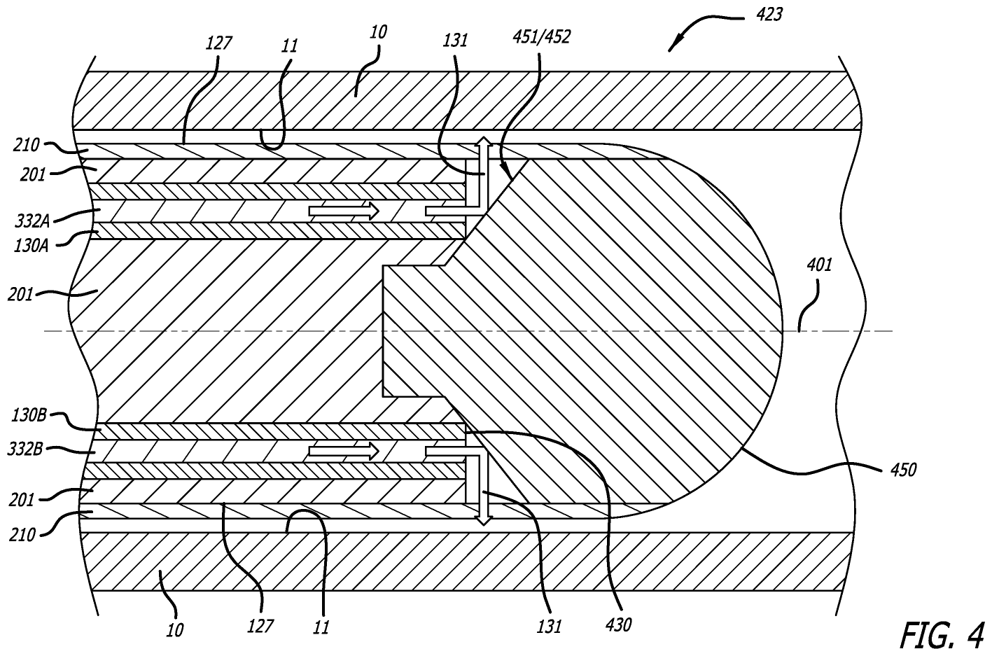

FIG. 4 is a detail cross-sectional view of another embodiment of a disinfection zone of the instrument FIG. 1. A disinfection zone 423 is shown in conjunction with the medical device 10. In this embodiment, the light 131 is reflected by a plug 450 including a cone shaped surface 451 having a reflecting material 452 disposed thereon. The plug 450 is coupled with the core 201 at its distal end 430. The coned shaped reflecting surface 451 is disposed coincident with the core 201 along the central axis 401. Each of the optical fibers 130 such as the optical fibers 130A, 130B shown in FIG. 4, are terminated at the distal end 430 in such a way that the light 131 propagating along the optical cores 332A, 332B exits the optical fibers 130 in a direction toward the reflecting surface 451. Upon reflection, the light 131 from each optical fiber 130 is directed radially outward where it propagates through the sheath 210 before impinging onto the inside surface 11. In some embodiments, the cone shaped reflecting surface 451 includes a single conical reflecting facet. In other embodiments, the cone shaped reflecting surface 451 includes multiple reflecting facets, such as a single facet for each optical fiber 130.

In some embodiments, the plug 450 may be attached directly to the core 201 via an attachment mechanism, such as bonding for example. In other embodiments, the plug 450 may be secured to the sheath 210.

Using the disinfection system 100 may include all or subset of the following steps or processes to disinfect the inside surface 11 of the tubular medical device 10. The medical device 10 may be inserted into the patient or separated from the patient. The clinician may choose an instrument 120 from a plurality instruments 120 of different sizes in accordance with the specific medical device 10. The clinician may couple the instrument 120 with the light source 110. The clinician may insert the instrument 120 into medical device 10 and advance the instrument 120 to a desired position so that the disinfecting zone 123 is positioned adjacent a portion of the medical device 10 to be disinfected. The clinician may activate the light source 110 to provide UV light 131 to the inside surface 11 of the medical device 10. The clinician may adjust the position of the instrument 120 relative to the medical device 10 to provide UV light 131 to another portion of the device 10. The clinician may repeatingly adjust the position until all desired portions of the device have been disinfected. The clinician may deactivate the light source and remove the instrument 120 from the device 10. In some embodiments, the clinician may activate the light source 110 only after inserting the instrument 120 into the device 10. Similarly, in some embodiments, the clinician may remove the instrument 120 from the device 10 only after deactivating the light source 110. The clinician may decouple the instrument 120 from the light source 110. The clinician may grasp the handle 124 to manipulate the instrument 120 during use.

In some embodiments, the use may activate a timer 13 of the system 100 and adjust the position of the instrument 120 upon receiving an alert from the timer 113. In some embodiments, the clinician may insert the instrument 120 into the medical device 10 so that a defined graduation mark 129 on the instrument 120 is disposed adjacent a proximal end of the medical device. The clinician may adjust the position of the instrument 120 relative to the medical device 10 so that a different graduation mark 129 is disposed adjacent the proximal end of the medical device 10.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for disinfecting a medical device, comprising:
an elongate instrument comprising a plurality of optical fibers extending along a length of the instrument from a proximal end to a disinfection zone at a distal end; and
a light source operatively coupled with the instrument at the proximal end, such that light from the light source propagates distally along the optical fibers, wherein the elongate instrument is configured to redirect the light, propagating along the optical fibers, radially outward from the instrument.

2. The system of claim 1, where the instrument is configured for insertion into a medical device.

3. The system of claim 2, wherein the medical device is an elongate tubular medical device.

4. The system of claim 3, wherein the medical device is a catheter.

5. The system of claim 4, wherein the medical device is a vascular catheter.

6. The system of claim 1, wherein the instrument is configured for insertion into the medical device while the medical device is inserted within a patient body.

7. The system of claim 1, wherein the instrument defines a circular cross section.

8. The system of claim 1, wherein the instrument comprises a conical reflective surface within the disinfection zone.

9. The system of claim 8, wherein each optical fiber comprises the reflective surface within the disinfection zone.

10. The system of claim 8, wherein the reflective surface is configured to direct fiber optic light radially outward from the instrument.

11. The system of claim 1, wherein a wavelength range of the light extends only between 100 nm and 400 nm.

12. The system of claim 1, wherein the instrument comprises a sheath extending along the length.

13. The system of claim 12, wherein the sheath comprises a material transparent to ultra-violet light.

14. The system of claim 1, wherein the instrument comprises indicia disposed on the instrument along a least a portion of the length.

15. The system of claim 14, wherein the indicia comprise graduation marks.

16. The system of claim 1, further comprising a timer configured to provide an alert at the conclusion of a defined time period.

17. The system of claim 1, wherein the instrument comprises a handle disposed at the proximal end, the handle configured for manipulation of the instrument by a clinician.

18. The system of claim 1, further comprising an interconnect extending between the instrument and the light source, the interconnect comprising interconnecting optical fibers to facilitate propagation of the light between the light source and the instrument.

19. An elongate instrument for disinfecting a medical device, comprising:
a plurality of optical fibers extending along a length of the instrument from a proximal end to a disinfection zone at a distal end, the optical fibers configured to propagate a light along the instrument;
one or more reflective surfaces located within the disinfection zone, the reflective surfaces configured to direct the light radially outward from the instrument; and
a connector at the proximal end configured to couple with a light source.

20. The instrument of claim 19, where the instrument is configured for insertion into a medical device.

21. The instrument of claim 20, wherein the medical device is a tubular medical device.

22. The instrument of claim 21, wherein the medical device is a catheter.

23. The instrument of claim 22, wherein the medical device is a vascular catheter.

24. The instrument of claim 19, wherein the instrument is configured for insertion into the medical device while the medical device is inserted within a patient body.

25. The instrument of claim 19, wherein the instrument defines a circular cross section.

26. The instrument of claim 19, further comprising:
a core extending between the proximal end and the disinfection zone; and
a sheath extending between the proximal end and the disinfection zone, the sheath disposed over the core so as to cover the core.

27. The instrument of claim 26, wherein the sheath comprises a material transparent to ultra-violet light.

28. The instrument of claim 26, wherein:
the optical fibers are disposed within the core, and
the optical fibers are disposed adjacent a circumferential surface of the core.

29. The instrument of claim 19, further comprising a plug coupled to the core at a distal end of the core, the plug having a reflective conical surface defining the one or more reflective surfaces.

30. The instrument of claim 19, wherein:
each optical fiber comprises a cavity extending laterally across an optical core of the optical fiber, the optical core configured to propagate the light along the optical fiber, and
a wall of the cavity is a reflective surface configured to direct the light from the optical core radially outward from the instrument.

31. The instrument of claim 30, wherein:
the core comprises one or more notches disposed on the circumferential surface along the disinfection zone,
the notches extend inward to the optical fibers, and
the notches are configured for passage of light therethrough.

32. The instrument of claim 19, further comprising graduation marks disposed on the instrument along a least a portion of the length.

33. The instrument of claim 19, further comprising a handle disposed at the proximal end, the handle configured for manipulation of the instrument by a clinician.

34-41. (canceled)
